(19) United States
(12) Patent Application Publication
(10) Pub. No.: US 2022/0218738 A1
(43) Pub. Date: Jul. 14, 2022

(54) POTASSIUM-BINDING AGENTS FOR USE IN HEMODIALYSIS PATIENTS

(71) Applicant: AstraZeneca AB, Sodertalje (SE)

(72) Inventors: Jenny JONASSON, Belmont, MA (US); Nicolas GUZMAN, Gaithersburg, MD (US)

(21) Appl. No.: 17/593,127

(22) PCT Filed: Mar. 12, 2020
(86) PCT No.: PCT/EP2020/056595

§ 371 Date: Sep. 9, 2021

Related U.S. Application Data
(60) Provisional application No. 62/817,817, filed on Mar. 13, 2019.

(51) Int. Cl. A61K 31/78; A61K 33/24; A61P 3/12

(52) U.S. Cl. CPC A61K 31/78; A61P 3/12; A61K 33/24

(57) ABSTRACT

The present invention relates to the use of potassium-binding agents that are formulated to remove toxins, e.g., potassium ions, from the gastrointestinal tract at an elevated rate, without causing undesirable side effects, in hemodialysis patients. The compositions exhibit desired characteristics for the long term administration to treat or prevent the relapse or occurrence of certain conditions, for example hyperkalemia.

### TECHNICAL FIELD

The present invention relates to the use of potassium-binding agents that are formulated to remove toxins, e.g., potassium ions, from the gastrointestinal tract at an elevated rate, without causing undesirable side effects, in hemodialysis patients. The compositions exhibit desired characteristics for the long term administration to treat or prevent the relapse or occurrence of certain conditions, for example hyperkalemia.

### BACKGROUND

Acute hyperkalemia is a serious life-threatening condition resulting from elevated serum potassium levels. Potassium is a ubiquitous ion, involved in numerous processes in the human body. It is the most abundant intracellular cation and is critically important for numerous physiological processes, including maintenance of cellular membrane potential, homeostasis of cell volume, and transmission of action potentials. Its main dietary sources are vegetables (tomatoes and potatoes), fruit (oranges, bananas) and meat. The normal potassium levels in plasma are between 3.5-5.0 mmol/l with the kidney being the main regulator of potassium levels. The renal elimination of potassium is passive (through the glomeruli) with active reabsorption in the proximal tubule and the ascending limb of the loop of Henle. There is active excretion of potassium in the distal tubules and the collecting duct, both of which processes are controlled by aldosterone. Increased extracellular potassium levels result in depolarization of the membrane potential of cells. This depolarization opens some voltage-gated sodium channels, but not enough to generate an action potential. After a short period of time, the open sodium channels inactivate and become refractory, increasing the threshold to generate an action potential. This leads to impairment of the neuromuscular-, cardiac- and gastrointestinal organ systems, and this impairment is responsible for the symptoms seen with hyperkalemia. Of greatest concern is the effect on the cardiac system, where impairment of cardiac conduction can lead to fatal cardiac arrhythmias such as asystole or ventricular fibrillation. Because of the potential for fatal cardiac arrhythmias, hyperkalemia represents an acute metabolic emergency that must be immediately corrected.

Hyperkalemia may develop when there is excessive production of serum potassium (oral intake, tissue breakdown). Ineffective elimination, which is the most common cause of hyperkalemia, can be hormonal (as in aldosterone deficiency), pharmacologic (treatment with ACE-inhibitors or angiotensin-receptor blockers) or, more commonly, due to reduced kidney function or advanced cardiac failure. The most common cause of hyperkalemia is renal insufficiency, and there is a close correlation between degree of kidney failure and serum potassium (S-K) levels. In addition, a number of different commonly used drugs cause hyperkalemia, such as ACE-inhibitors, angiotensin receptor blockers, potassium-sparing diuretics (e.g. amiloride, spironolactone), NSAIDs (such as ibuprofen, naproxen, celecoxib), heparin and certain cytotoxic and/or antibiotic drugs (such as cyclosporin and trimethoprim). Finally, beta-receptor blocking agents, digoxin or succinylcholine are other well-known causes of hyperkalemia. In addition, advanced degrees of congestive heart disease, massive injuries, burns or intravascular hemolysis cause hyperkalemia, as can metabolic acidosis, most often as part of diabetic ketoacidosis.

Symptoms of hyperkalemia are somewhat non-specific and generally include malaise, palpitations and muscle weakness or signs of cardiac arrhythmias, such as palpitations, brady-tachycardia or dizziness/fainting. Often, however, the hyperkalemia is detected during routine screening blood tests for a medical disorder or after severe complications have developed, such as cardiac arrhythmias or sudden death. Diagnosis is obviously established by S-K measurements.

Treatment depends on the S-K levels. In milder cases (S-K between 5-6.5 mmol/l), acute treatment with a potassium binding resin (Kayexalate®), combined with dietary advice (low potassium diet) and possibly modification of drug treatment (if treated with drugs causing hyperkalemia) is the standard of care; if S-K is above 6.5 mmol/l or if arrhythmias are present, emergency lowering of potassium and close monitoring in a hospital setting is mandated. The following treatments are typically used following emergency lowering of potassium: Kayexalate®, a resin that binds potassium in the intestine and hence increases fecal excretion, thereby reducing S-K levels. However, as Kayexalate® has been shown to cause intestinal obstruction and potential rupture. Further, diarrhea needs to be simultaneously induced with treatment. These factors have reduced the palatability of treatment with Kayexalate®.

Patiromer (Veltassa), a cross-linked polymer of 2-fluoroacrylic acid with divinylbenzenes and 1,7-octadiene used in form of its calcium salt and with sorbitol, a combination called patiromer sorbitex calcium.

Sodium Zirconium Cyclosilicate (Lokelma or SZC), a zirconium silicate microporous ion exchanger.

Insulin IV (+glucose to prevent hypoglycemia), which shifts potassium into the cells and away from the blood.

Calcium supplementation. Calcium does not lower S-K, but it decreases myocardial excitability and hence stabilizes the myocardium, reducing the risk for cardiac arrhythmias.

Bicarbonate. The bicarbonate ion will stimulate an exchange of potassium for sodium, thus leading to stimulation of the sodium-potassium ATPase, dialysis (in severe cases).

The kidney plays a major role in eliminating potassium. Patients with end-stage renal disease (ESRD) have reduced renal potassium excretion ability which frequently leads to hyperkalemia (S-K>5.1 mmol/L). These patients depend on the administration of renal replacement therapies (e.g. hemodialysis including low potassium dialysates as necessary), dietary potassium restriction, and occasionally the use of oral potassium binding resins to maintain serum potassium levels in a physiologic range (Clin J Am Soc Nephrol 11: 90-100, 2016, Clin J Am Soc Nephrol 2: 999-1007, 2007). High serum potassium can lead to ventricular arrhythmias and cardiac death. Recent studies have shown that among patients with ESRD on hemodialysis therapy, S-K>5.6 mmol/L is associated with increased mortality, both all-cause and cardiovascular, compared to a referent category of S-K levels between 4.6 to 4.99 mmol/L (Clin J Am Soc Nephrol 2: 999-1007, 2007, Am J Nephrol 44:179-186, 2016). In addition, sudden cardiac death (SCD) is the leading cause of death in hemodialysis patients. In the United States Renal Data System (USRDS) database, 26.9% of all-cause mortalities in prevalent dialysis patients between 2009 and 2011 were attributed to cardiac arrest or arrhythmias. The incidence of SCD in hemodialysis patients was 49.2 per 1000 patient-years in 2011, which is much higher than that of the general population (PLoS One. 2015 Oct. 6; 10(10): e0139886. doi:10.1371/journal.pone.0139886). Hemodialysis patients have high pre-dialysis potassium conc. Those patients are usually managed with dialysis on Monday, Wednesday and Friday. After dialysis, serum K rebounds rapidly and becomes hyperkalemic again before the next cycle dialysis. Pre-dialysis hyperkalemia and low potassium dialysate are associated with increased risk of sudden cardiac arrest, sudden cardiac death and CV mortality.

Hyperkalemia is considered an important risk factor for arrhythmias and SCD. This condition is also independently associated with greater short-term risk of hospitalization and emergency department visits, and with greater hospital costs (Am J Kidney Dis 70:21-29 2017). Hence prevention and treatment of hyperkalemia in hemodialysis patients are of paramount importance.

Currently the only universally accepted option for the treatment of hyperkalemia in patients with ESRD is dialysis including low potassium dialysates as necessary (hemo- or peritoneal dialysis, and hemodiafiltration). Despite dialysis the prevalence of hyperkalemia remains high in this population with a prevalence as high as 62.9 per 100 patient-months at the end of the long interdialytic interval (Am J Nephrol 44:179-186, 2016). In this latter study hyperkalemia was defined as a pre-dialysis serum potassium greater than 5.5 mmol/L and its presence was associated with increased all-cause mortality. Although potassium-binding resins are used in some instances to treat hyperkalemia in dialysis patients, these agents have not been systematically studied, are not universally used, and have no specific indications in this population.

### SUMMARY

The present disclosure relates to the administration of potassium-binding agents to hemodialysis patients, hereby maintaining normokalemia during interdialytic intervals.

### LIST OF FIGURES

FIG. 1 Study flowchart

FIG. 2 Schedule of assessments—treatment and follow-up phase

FIG. 3 Analysis of proportion of responders

FIG. 4 Effect on pre- and post-dialysis potassium concentrations

### DETAILED DESCRIPTION

In one embodiment, the present disclosure involves administration of a suitable dose of a potassium-binding agent to a hemodialysis patient.

In one embodiment, the present disclosure involves administration of a suitable dose of microporous zirconium silicate to a hemodialysis patient.

In one embodiment, the present disclosure involves administration of a suitable dose of Sodium Zirconium Cyclosilicate to a hemodialysis patient.

In one embodiment, the present disclosure involves administration of a suitable dose of 2-fluoroacrylate-divinylbenzene-1,7-octadiene copolymer crosslinked in the salt or acid form to a hemodialysis patient.

In a further embodiment, the present disclosure involves administration of a suitable dose of patiromer sorbitex calcium to a hemodialysis patient.

In another embodiment, the present disclosure involves administration of a suitable dose of Sodium Zirconium Cyclosilicate to a hemodialysis patient pre-dialysis, i.e administration on non-dialysis days.

In one embodiment, the dosage of potassium-binding agent may range from 1-30 g, preferably 5-15 g, more preferably 5 g.

In another embodiment, the dosage of potassium-binding agent may range from 1-30 g, preferably 5-15 g, more preferably 10 g.

In another embodiment, the dosage of potassium-binding agent may range from 1-30 g, preferably 10-20 g, more preferably 15 g.

In another embodiment, the present disclosure involves administration of 5 grams of Sodium Zirconium Cyclosilicate to a hemodialysis patient pre-dialysis, i.e administration on non-dialysis days.

In another embodiment, the present disclosure involves administration of 10 grams of Sodium Zirconium Cyclosilicate to a hemodialysis patient pre-dialysis, i.e administration on non-dialysis days.

In another embodiment, the present disclosure involves administration of 15 grams of Sodium Zirconium Cyclosilicate to a hemodialysis patient pre-dialysis, i.e administration on non-dialysis days.

The use of zirconium silicate or titanium silicate microporous ion exchangers to remove toxic cations and anions from blood or dialysate is described in U.S. Pat. Nos. 6,579,460, 6,099,737, 6,332,985 and U.S. 2004/0105895, each of which is incorporated herein in their entirety. Additional examples of microporous ion exchangers are found in U.S. Pat. Nos. 6,814,871, 5,891,417, and 5,888,472, each of which is incorporated herein in their entirety.
Certain zirconium silicate compositions may exhibit undesirable effects when utilized in vivo for the removal of potassium in the treatment of hyperkalemia. Specifically, the inventors found that administration of zirconium silicate molecular sieve compositions is associated with an incidence of mixed leukocyte inflammation, minimal acute urinary bladder inflammation and the observation of unidentified crystals in the renal pelvis and urine in animal studies, as well as an increase in urine pH. These problems were addressed by controlling particle size and sodium content of the zirconium silicate compositions. See U.S. Pat. Nos. 8,802,152 and 8,808,750, each of which is incorporated herein in their entirety.
Further, certain zirconium silicate compositions have had issues with crystalline impurities and undesirably low cation exchange capacity. The reduction of more soluble forms of zirconium silicate is important to reduce or eliminate the systemic absorption of zirconium or zirconium silicate. This issue was addressed by controlling production conditions in a way that essentially eliminates ZS-8 from the composition, resulting in undetectable levels of ZS-8. See U.S. Pat. No. 8,877,255.
Certain zirconium silicate compositions are useful for long term use, for example, in the treatment of conditions associated with elevated levels of serum potassium. The use of zirconium silicate compositions in long term treatment regimens requires careful control of impurities, particularly lead, in the composition. For example, the FDA sets the acceptance criteria for lead in compositions for extended use at 5 micrograms per day. Certain zirconium silicates produced using known methods in industrial quantities contain approximately 1 to 1.1 ppm or more of lead. Even when zirconium silicate was prepared in smaller batches at higher purity, the level of lead was found to be 0.6 ppm or more.
Because zirconium silicate treatments utilize doses ranging from 5 to 45 grams per day, reduction in the level of lead is necessary. Compositions of zirconium silicate having lead content within an acceptable range necessitated by the daily doses of zirconium silicate are disclosed in US2017/0151279A1.
Sodium Zirconium Cyclosilicate is a cation exchange composition comprising a zirconium silicate of formula (I):

AₚMₓZr₁₋ₓSiₙGe_{y}Oₘ (I)

where

A is a potassium ion, sodium ion, rubidium ion, cesium ion, calcium ion, magnesium ion, hydronium ion or mixtures thereof,

M is at least one framework metal, wherein the framework metal is hafnium (4+), tin (4+), niobium (5+), titanium (4+), cerium (4+), germanium (4+), praseodymium (4+), terbium (4+) or mixtures thereof,

“p” has a value from about 1 to about 20,

“x” has a value from 0 to less than 1,

“n” has a value from about 0 to about 12,

“y” has a value from 0 to about 12,

“m” has a value from about 3 to about 36 and 1≤n+y≤12,

wherein the composition exhibits a lead content below 0.6 ppm. Preferably, the lead content ranges from 0.1 and 0.6 ppm, more preferably from 0.3 to 0.5 ppm, and most preferably from 0.3 to 0.45 ppm. In one embodiment, the lead content is 0.38 ppm.

In addition to having a desired level of lead impurity, the composition may exhibit one or more properties that make it desirable as an orally ingested ion trap. In one aspect, the zirconium silicate composition may have a potassium exchange capacity exceeding 2.3 meq/g, preferably ranging from 2.3 to 3.5 meq/g, more preferably within the range of 3.05 and 3.35 meq/g, and most preferably about 3.2 meq/g. In one embodiment, 7% of the particles in the composition have a diameter less than 3 microns. In other embodiments less than 0.5% of the particles in the composition have a diameter less than 1 microns. Preferably, the sodium content is below 12% by weight, and more preferably 9% or less by weight. The zirconium silicate preferably exhibits an XRD diffractogram having the two highest peaks occur at approximately 15.5 and 28.9, with the highest peak occurring at 28.9. The material is preferably ZS-9, or predominately ZS-9, having a pH ranging from 7 to 9 and a potassium loading capacity between 2.7 and 3.7 mEq/g, and most preferably approximately 3.5.

### EXAMPLES

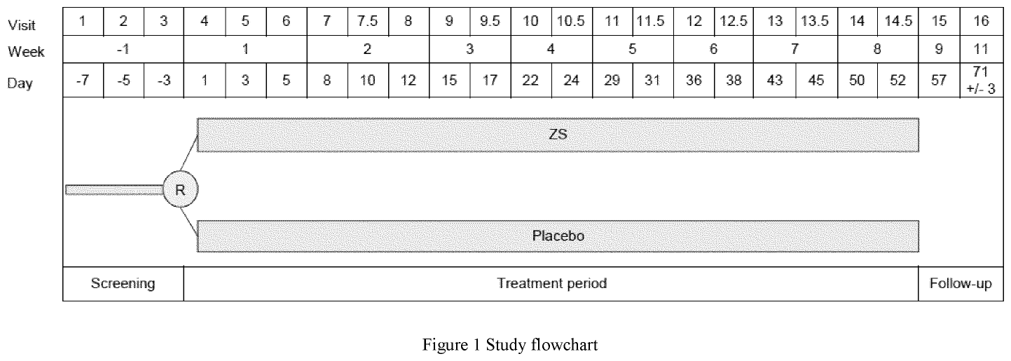

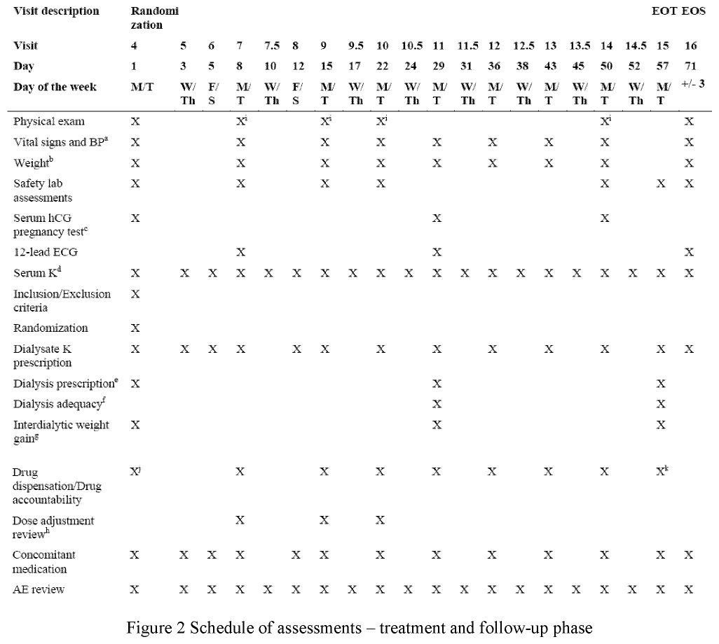

A Phase 3b, Multicenter, Prospective, Randomized, Double Blind, Placebocontrolled Study to Reduce Incidence of Pre-Dialysis Hyperkalemia with Sodium Zirconium Cyclosilicate (SZC) The study was carried out to evaluate the efficacy of Sodium Zirconium Cyclosilicate in the treatment of hyperkalemia in patients on hemodialysis. The study was designed to include approximately 180 patients with ESRD receiving maintenance hemodialysis treatments three times per week with an indication for treatment of hyperkalemia (FIG. 1). The study is a randomized, double-blind study with two treatment groups, SZC or placebo, and includes hemodialysis patients that have been on dialysis for a minimum of three months and receive treatment three times a week. Patients must have hemodialysis access consisting of an arteriovenous fistula, AV graft, or tunneled (permanent) catheter which is expected to remain in place for the entire duration of the study (FIG. 2).

The starting dose of SZC will be 5 g once daily on non-dialysis day and may be adjusted to a maximum of 15 g per non-dialysis day to maintain a pre-dialysis S-K between 4-5 mmol/L. SZC or placebo will be administered orally on non-dialysis days for a treatment period of eight weeks. Patients will be randomized (1:1) to double-blind treatment with either SZC or placebo, started at 5 g once daily on non-dialysis days, and titrated during a period of four weeks to achieve and maintain a pre-dialysis serum potassium between 4 and 5 mmol/L after the Long Inter-Dialytic Interval (LIDI). Maximum SZC dose is 15 g once daily on non-dialysis days. Treatment will be continued unchanged for an additional four week evaluation period to complete a total of 8 weeks. The primary benefit for patients randomized to SZC is expected to be the maintenance of normokalemia during the long interdialytic interval, potentially including the relief of associated signs and symptoms and an improved quality of life.

### Inclusion Criteria

For inclusion in the study patients should fulfil the following criteria:
1. Provision of informed consent prior to any study specific procedures.
2. Female or male aged >18 years at screening Visit 1. For patients aged <20 years and enrolled in Japan, a written informed consent should be obtained from the patient and his or her legally acceptable representative.
3. Receiving hemodialysis (or hemodiafiltration) three times a week for treatment of endstage renal disease (ESRD) for at least three months before randomization.
4. Patients must have hemodialysis access consisting of an arteriovenous fistula, AV graft, or tunneled (permanent) catheter which is expected to remain in place for the entire duration of the study.
5. Pre-dialysis S-K>5.4 mmol/L after long inter-dialytic interval and >5.0 mmol/L after one short inter-dialytic interval during screening.
6. Prescribed dialysate K concentration ≤3 mmol/L during screening
7. Sustained Qb≥200 ml/min and spKt/V≥1.2 (or URR≥63) on stable hemodialysis/hemodiafiltration prescription during screening with prescription (time, dialyzer, blood flow [Qb], dialysate flow rate [Qd] and bicarbonate concentration) expected to remain unchanged during study.
8. Heparin dose (if used) must be stable during screening and expected to be stable during the study.
9. Subjects must be receiving dietary counseling appropriate for ESRD patients treated with hemodialysis/hemodiafiltration as per local guidelines, which includes dietary potassium restriction.

### Exclusion Criteria

Patients should not enter the study if any of the following exclusion criteria are fulfilled:
1. Involvement in the planning and/or conduct of the study.
2. Hemoglobin <9 g/dL on screening (as assessed on Visit 1).
3. Lack of compliance with hemodialysis prescription (both number and duration of treatments) during the two-week period preceding screening (100% compliance required).
4. Patients treated with sodium polystyrene sulfonate (SPS, Kayexalate, Resonium), calcium polystyrene sulfonate (CPS, Resonium calcium) or patiromer (Veltassa) within 7 days before screening or anticipated in requiring any of these agents during the study.
5. Myocardial infarction, acute coronary syndrome, stroke, seizure or a thrombotic/thromboembolic event (e.g., deep vein thrombosis or pulmonary embolism, but excluding vascular access thrombosis) within 12 weeks prior to randomization.
6. Laboratory diagnosis of hypokalemia (S-K<3.5 mmol/L), hypocalcemia (Ca<8.2 mg/dL; for Japan hypocalcemia is defined as albumin-corrected Ca<8.0 mg/dL), hypomagnesemia (Mg<1.7 mg/dL) or severe acidosis (serum bicarbonate 16 mEq/L or less) in the four weeks preceding randomization.
7. Pseudohyperkalemia secondary to hemolyzed blood specimen (this situation is not considered screening failure, sampling or full screening can be postponed to a later time as applicable).
8. Severe leukocytosis (>20×109/L) or thrombocytosis (>450×109/L) during screening.
9. Polycythemia (Hb>14 g/dL) during screening.
10. Diagnosis of rhabdomyolysis during the four weeks preceding randomization.
11. Patients treated with lactulose, xifaxan (rifaximin) or other non-absorbed antibiotics for hyperammonemia within seven days prior to the first dose of study drug.
12. Patients unable to take oral SZC drug mix.
13. Scheduled date for living donor kidney transplant.
14. Patients with a life expectancy of less than six months.
15. Female patients who are pregnant or breastfeeding.
16. Females of childbearing potential, unless using contraception as detailed in the protocol or sexual abstinence.
17. Known hypersensitivity or previous anaphylaxis to SZC or to components thereof 18. Participation in another clinical study with an investigational product during the last one month before screening.
19. Any medical condition, including active, clinically significant infection, that in the opinion of the investigator or Sponsor may pose a safety risk to a patient in this study, which may confound safety or efficacy assessment and jeopardize the quality of the data, or may interfere with study participation.
20. Presence of cardiac arrhythmias or conduction defects that require immediate treatment.
21. History of alcohol or drug abuse within two years prior to randomization.
22. Previous randomization in the present study.

### Efficacy Assessments

### Serum Potassium Measurements

Serum potassium levels (S-K) will be measured using i-STAT device (Point-Of-Care analyser) and central laboratory (c-Lab).
Potassium samples will be analysed locally using i-STAT devices for the purpose of dose titration and treatment control. In the event that hemolysis or other artefacts are suspected based on the reported i-STAT result the sample may be re-drawn to confirm the result.

### Dialysate Potassium Concentration Prescription and Potassium Levels

For pre-dialysis serum potassium concentrations <4 mmol/L, subsequent adjustments will be made in accordance to locally accepted clinical practice patterns and guided by the investigator's clinical judgment. For centers that adopt the clinical practice of modifying the prescribed dialysate potassium concentration when the pre-dialysis serum potassium concentration decreases, if pre-dialysis serum K is below 4 mmol/L the dialysate K concentration should be increased by 0.5 or 1 mmol/L according to standard of care, e.g. increase dialysate K from 1K to 1.5 or 2K, from 2K to 2.5 or 3K, or from 3K to 3.5 or 4K.
SZC or placebo will be suspended in 45 ml of water and administered orally on non-dialysis days for a treatment period of eight weeks. The initial SZC dose will be 5 g once daily and may be adjusted to a maximum of 15 g per non-dialysis day to maintain a pre-dialysis S-K between 4-5 mmol/L.
All dose adjustments will be based on pre-dialysis S-K values measured by i-STAT.
Management of dialysis prescription will be according to local clinical pattern practices.
During the first four weeks of the treatment period, the SZC dose should be adjusted if the predialysis potassium value after the long inter-dialytic interval is >5.0 mmol/L (one weekly dose adjustment). For patients taking 5 g on non-dialysis days, the dose should be increased to 10 g on non-dialysis days. For patients taking 10 g, the dose should be increased to 15 g on non-dialysis days. During the first four weeks of the treatment period, both pre- and post-dialysis serum potassium concentrations should be evaluated.
For pre-dialysis serum potassium concentrations <4 mmol/L, subsequent adjustments will be made in accordance to locally accepted clinical practice patterns and guided by the investigator's clinical judgment.
For sites that adopt the clinical practice of modifying the prescribed dialysate potassium concentration when the pre-dialysis serum potassium concentration decreases, if pre-dialysis S-K is below 4 mmol/L the dialysate K concentration should be increased by 0.5 or 1 mmol/L according to standard of care, e.g. increase dialysate K from 1K to 1.5 or 2K, from 2K to 2.5 or 3K, or from 3K to 3.5 or 4K. If dialysate K concentration cannot be increased further (e.g. patient already using 4K dialysate bath), the dose of SZC can be decreased by 5 g or held if the patient is already taking the minimum dose (5 g).
For sites where local clinical practice does not include increasing the dialysate K concentration when pre-dialysis serum K falls, the dose of SZC can be decreased by 5 g or held if the patient is already taking the minimum dose (5 g). If during the treatment phase (initial four weeks) the dose of SZC has been reduced or held and the pre-dialysis potassium value after the next long interdialytic interval is above 5.0 mmol/L, every effort should be made to increase the dose by 5 g or restart SZC 5 g if it was held.
After the first four weeks, no additional adjustments of SZC dose or dialysate potassium concentration should be made unless in the judgement of the principal investigator there is a compelling medical need to treat an abnormal serum potassium concentration, i.e. severe hyperkalemia or hypokalemia with clinical manifestations. If such an event were to occur the appropriate SZC dose adjustment (increase or reduction) can be made with documentation of the event.
In the case of hyperkalemia with clinical manifestations deemed to require urgent treatment, rescue therapy defined as any intervention consistent with local practice patterns to reduce serum K can be administered followed by the appropriate SZC dose adjustment and proper documentation of the event. During the last four weeks of the treatment period, both pre and post-dialysis serum potassium concentrations will continue to be evaluated. It is recommended that the dietary regimen is maintained unchanged during the duration of the study.

### Results

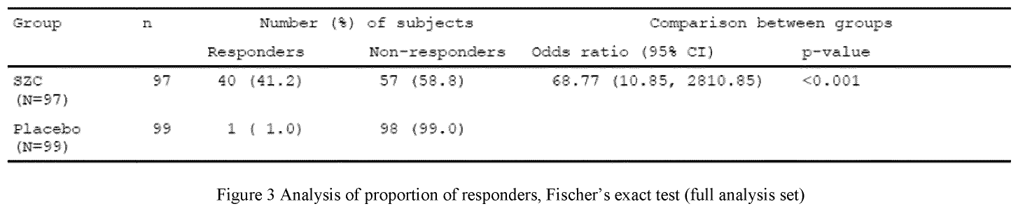

97 patients were randomised onto SZC and 99 patients were randomized into the placebo group. Except for one patient in the SZC group, all randomized patients received treatment. The primary outcome measure of the study is defined as the proportion of patients who maintain a pre-dialysis serum potassium between 4.0-5.0 mmol/L on 3 out of 4 dialysis treatments following the long interdialytic interval (LIDI), and not receiving rescue therapy, during during the evaluation period (last 4 weeks). The analysis was done using ITT (intention to treat) principle. All randomized patients are in the analysis, even those who did not receive treatment. This means that, for example, difference in treatment discontinuation between treatment arms could have an influence on the result. Even if a patient has missing data, they are included as non-responders (FIG. 3). After the dose-adjustment period (the starting dose was 5 g), 37%, 43% and 19% were on SZC 5 g, 10 g and 15 g, respectively. One patient was down-titrated to 0 g.

The number of patients adverse events was balanced between treatment groups, 40 in the SZC group and 46 in the placebo group. Of these, 7 in the SZC group and 8 in the placebo group were regarded as serious adverse event, including a death in the SZC group, which was judged not to be related to the investigational product. There were 10 patients with pre-dialysis hypokalemia (defined as serum K<3.5 mmol/L, five in each treatment group.

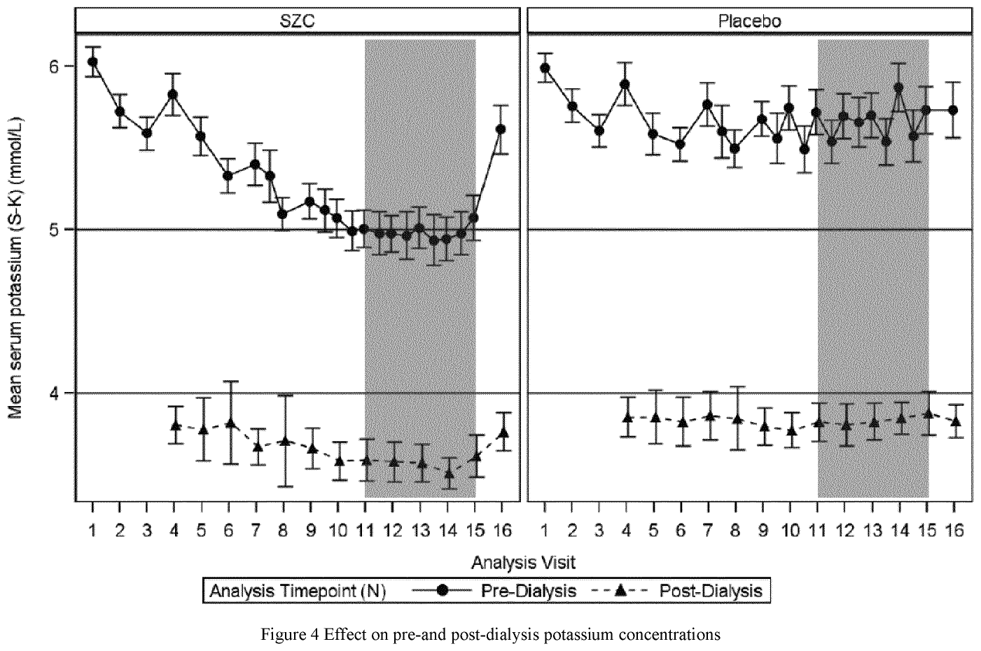

Pre-dialysis mean serum-K decrease during the dose adjustment period in the SCZ group is stable in the evaluation period and increase after the follow-up period. In the placebo group the pre-dialysis mean serum-K is stable over the treatment period. The post-dialysis mean serum-K shows similar patterns, though less pronounced (FIG. 4).

Mean K-shift is smaller in the SZC group compared to the placebo group starting at visit 9 through the evaluation period. The mean K-shift in the placebo group was about 1.9 mmol/L. The mean K-shift in SZC group was 1.4-1.5 mmol/L between visits 9 and 15.

Mean K-gradient is smaller in the SZC group compared to the placebo group starting visit 8 through the evaluation period. The mean K-gradient in placebo group was about 3.5 mmol/L. The mean K-gradient in the SZC group was 2.7-2.9 mmol/L between visits 8 to 15.

The proportion of responders is statistically significantly higher in SZC compared to placebo, with 41.2% responders in the SZC group compared to 1.0% in the placebo group. (FIG. 4: The bars represent 2*Standard Deviation (mean)). Treatment with SZC raised no safety concerns.

1. A method for treatment of hyperkalemia in a hemodialysis patient comprising administering a potassium-binding agent to a patient in need thereof.

2. The method of claim 1, where the potassium-binding agent is a microporous zirconium silicate.

3. The method of claim 1, where the potassium-binding agent is Sodium Zirconium Cyclosilicate.

4. The method of claim 1, where the potassium-binding agent is administered on non-dialysis days.

5. The method of claim 3, where the potassium-binding agent is administered in a 5 gram dose.

6. The method of claim 3, where the potassium-binding agent is administered in a 10 gram dose.

7. The method of claim 3, where the potassium-binding agent is administered in a 15 gram dose.

8. The method of claim 1, where the potassium-binding agent is administered on non-dialysis days.

9. The method of claim 1, where the potassium-binding agent is a 2-fluoroacrylate-divinylbenzene-1,7-octadiene copolymer crosslinked in the salt or acid form.

10. The method of claim 1, where the 2-fluoroacrylate-divinylbenzene-1,7-octadiene copolymer crosslinked in the salt or acid form is patiromer sorbitex calcium.
